(19) United States
(12) Patent Application Publication
(10) Pub. No.: US 2012/0010285 A1
(43) Pub. Date: Jan. 12, 2012

(54) AGENT FOR PROMOTING ENERGY CONSUMPTION

(75) Inventors: Takatoshi Murase, Tochigi (JP); Koichi Misawa, Tochigi (JP); Yoshihiko Minegishi, Tochigi (JP); Noriyasu Ota, Tochigi (JP); Hideo Ohminami, Tochigi (JP)

(73) Assignee: KAO CORPORATION, Tokyo (JP)

(21) Appl. No.: 13/257,479

(22) PCT Filed: Mar. 17, 2010
(86) PCT No.: PCT/JP2010/001902

§ 371 Date: Sep. 19, 2011

(30) Foreign Application Priority Data
Mar. 18, 2009 (JP) 2009-066623

(51) Int. Cl. A61K 31/216; A61P 3/00

(52) U.S. Cl. 514/533

(57) ABSTRACT

Provision of a pharmaceutical product, a quasi-drug, a food, a beverage, a pet food, a feed, and others which are highly safe, have excellent action of promoting energy consumption, promoting fat burning, promoting carbohydrate burning, or improving an effect of exercise, and which are effective for the prevention or amelioration of obesity or metabolic syndrome or the improvement of motor functions.

An agent for promoting energy consumption, an agent for promoting fat burning, an agent for promoting carbohydrate burning, an agent for improving an effect of exercise, an agent for improving motor functions, an agent for inhibiting acetyl-CoA carboxylase 2, and an agent for inhibiting pyruvate dehydrogenase kinase 4 containing chlorogenic acids or salts thereof as an active ingredient.

### FIELD OF THE INVENTION

The present invention relates to an agent for promoting energy consumption, an agent for promoting fat burning, an agent for promoting carbohydrate burning, an agent for improving the effect of exercise, an agent for improving motor functions, an agent for inhibiting acetyl-CoA carboxylase 2 (ACC2), and an agent for inhibiting pyruvate dehydrogenase kinase 4 (PDK4).

### BACKGROUND OF THE INVENTION

Obesity refers to a condition in which an individual weighs more than usual as a result of excessive accumulation of energy intake from carbohydrate, fat, and the like in the form of fat under the skin or around the viscera. Also, recently, a concept of metabolic syndrome has been the focus of attention. Metabolic syndrome refers to a condition associated with underlying excessive accumulation of visceral fat (obesity), in which one is prone to develop arteriosclerosis complicated with abnormal glucose tolerance (hyperglycemia), high triglyceride (hyperlipidemia), and hypertension, namely, a condition in which one faces an increased risk of developing lifestyle disease.

In Japan, according to the diagnostic criteria for metabolic syndrome published in 2005, males having a waist circumference of 85 cm or more or females having a waist circumference of 90 cm or more who satisfy two or more items of the following three items, which are (1) having blood triglyceride of 150 mg/dl or more or HDL cholesterol of less than 40 mg/dl, (2) having hyperglycemia (fasting blood glucose of 110 mg/dl or more), and (3) having hypertension (130/85 mHg or more), are determined to have metabolic syndrome. Further, according to the investigation conducted by Ministry of Health, Labour and Welfare based on the above diagnostic criteria (May, 2006), 13 million Japanese qualify as having metabolic syndrome, with the number reaching 27 million when those who are likely to develop metabolic syndrome are included.

An increase of people who qualify as suffering from obesity and metabolic syndrome may incur increased medical costs, and obesity and metabolic syndrome are becoming a huge issue not only in Japan but on the worldwide scale.

Because obesity is induced when the amount of energy intake exceeds the amount of energy consumed, in order to ameliorate obesity, a method of decreasing the amount of energy intake from fat, carbohydrate, and the like or a method for increasing the amount of energy consumption by promoting in vivo metabolism by some methods are possible. Therefore, improvements of dietary habit and exercise are considered as an effective method for the prevention and amelioration of obesity and metabolic syndrome.

As for compounds promoting energy consumption, caffeine, chili pepper, and the like which have a sympathetic nerve-activating action are known (Non Patent Documents 1 to 3). Specifically, caffeine in coffee is known to increase heat production (Non Patent Document 4). However, since not only that caffeine is known to have sympathomimetic action and various side effects such as a caffeine intoxication, a diuretic action, and the effect on the fetus but also for its bitterness, it is unsatisfactory in terms of practical utility, such as safety and palatability.

Also, capsiate has recently been discovered as a less pungent capsaicin analog, and its action of promoting energy consumption has been confirmed (Non Patent Document 5).

Meanwhile, although exercise activates energy metabolism mainly in muscle and increases energy consumption, and thus is an effective method for the prevention and amelioration of obesity and metabolic syndrome, performing regular exercise is practically difficult in today's society, which becomes a major cause of the growth of the population of obesity and metabolic syndrome. Accordingly, development of a substance and a method which are safe and available for continuous application and effectively promote energy consumption are demanded.

Also, if the action of exercise can be enhanced, it is considered to serve as effective means for the prevention and amelioration of obesity and metabolic syndrome. Although tea catechin has been reported as an ingredient enhancing the effect of exercise (Patent Document 1), virtually no other ingredients are known.

Further, activation of energy metabolism and promotion of carbohydrate and fat burning are considered to lead to the improvement of motor functions.

Chlorogenic acid (5-caffeoylquinic acid) is a kind of polyphenol contained in coffee and the like, which has been so far reported to have various physiological actions. For example, it is known that chlorogenic acid has a fatty acid synthase-inhibiting action (Non Patent Document 6), and coffee containing chlorogenic acid is known to have a blood glucose absorption-inhibiting action and a body weight-suppressing action (Non Patent Document 7).

Also, it is known that chlorogenic acids have a glucose-6-phosphatase-inhibiting action (Non Patent Document 8), a fat metabolism-activating action by activation of the peroxisome proliferator-activated receptor (PPAR), a preventive and ameliorating action on hyperinsulinemia and hyperleptinemia (Patent Document 2), and a hypotensive action by reduction of oxidative stress (Non Patent Document 9), 3,4-dichlorogenic acid, 3,5-dichlorogenic acid, and 3,4,5-trichlorogenic acid have a maltase-inhibiting action (Non Patent Document 10), and neochlorogenic acid and feruloylquinic acid have a carnitine palmitoyltransferase-activating action (Patent Document 3).

However, the previous findings as noted above are derived from the examination of enzyme activities, inhibitory actions of enzymes, and changes in the blood components, and not associated with energy consumption and the amount of fat or carbohydrate burned.

Coffee is reported to increase heat production as described above, and it is considered that caffeine, which is contained in abundance in coffee, is an active ingredient inducing heat production (Non Patent Document 4).

Therefore, whether or not chlorogenic acids or salts thereof have an action of promoting energy consumption, an action of promoting fat burning, an action of promoting carbohydrate burning, or an action of improving the effect of exercise remains unknown until now.

### PRIOR ART DOCUMENTS

### Patent Document

[Patent Document 1] U.S. Patent Application Publication No. 2006/0173070

[Patent Document 2] JP-A-2003-34636

[Patent Document 3] JP-A-2006-342145

### Non Patent Document

[Non Patent Document 1] Dulloo A G., Am. J. Clin. Nutr., 1989, 49 (1): 44 to 50.

[Non Patent Document 2] Kawada T., Proc. Soc. Exp. Biol. Med., 1986, 183 (2): 250 to 6.

[Non Patent Document 3] Diepvens K., Am. J. Physiol. Regul. Integr. Comp. Physiol., 2007, 292 (1): R77 to 85

[Non Patent Document 4] Greenberg J A., Am. J. Clin. Nutr., 84, 682 to 693, 2006

[Non Patent Document 5] Ohnuki K., Biosci. Biotechnol. Biochem., 2001, 65 (12): 2735 to 40

[Non Patent Document 6] Li B H., IUBMB. Life, 58 (1), 39 to 46, 2006

[Non Patent Document 7] Thom E., J. Int. Med. Res., 35 (6), 900 to 908, 2007

[Non Patent Document 8] Arion W J., Arch. Biochem. Biophys., 339 (2), 315 to 322, 1997

[Non Patent Document 9] Suzuki A., Hypertens. Res., 24 (6), 1065 to 1073, 2006

[Non Patent Document 10] Matsui T., Biol. Pharm. Bull., 27 (11), 1797 to 1803, 2004

### SUMMARY OF INVENTION

Therefore, the present invention relates to the following inventions.
1) An agent for promoting energy consumption containing chlorogenic acids or salts thereof as an active ingredient.
2) An agent for promoting fat burning containing chlorogenic acids or salts thereof as an active ingredient.
3) An agent for promoting carbohydrate burning containing chlorogenic acids or salts thereof as an active ingredient.
4) An agent for promoting fat burning and carbohydrate burning containing chlorogenic acids or salts thereof as an active ingredient.
5) An agent for improving the effect of exercise containing chlorogenic acids or salts thereof as an active ingredient.
6) An agent for improving motor functions containing chlorogenic acids or salts thereof as an active ingredient.
7) An agent for inhibiting acetyl-CoA carboxylase 2 containing chlorogenic acids or salts thereof as an active ingredient.
8) An agent for inhibiting pyruvate dehydrogenase kinase 4 containing chlorogenic acids or salts thereof as an active ingredient.
9) An agent for inhibiting acetyl-CoA carboxylase 2 and pyruvate dehydrogenase kinase 4containing chlorogenic acids or salts thereof as an active ingredient.
10) A coffee beverage composition and a packaged beverage containing 0.01 to 1% by weight of chlorogenic acids or salts thereof for the promotion of energy consumption, fat burning or carbohydrate burning, or the improvement of the effect of exercise or the improvement in motor functions.
11) Use of chlorogenic acids or salts thereof for the production of an agent for promoting energy consumption.
12) Use of chlorogenic acids or salts thereof for the production of an agent for promoting fat burning.
13) Use of chlorogenic acids or salts thereof for the production of an agent for promoting carbohydrate burning.
14) Use of chlorogenic acids or salts thereof for the production of an agent for promoting fat burning and carbohydrate burning.
15) Use of chlorogenic acids or salts thereof for the production of an agent for improving the effect of exercise.
16) Use of chlorogenic acids or salts thereof for the production of an agent for improving motor functions.
17) Use of chlorogenic acids or salts thereof for the production of an agent for inhibiting acetyl-CoA carboxylase 2.
18) Use of chlorogenic acids or salts thereof for the production of an agent for inhibiting pyruvate dehydrogenase kinase 4.
19) Use of chlorogenic acids or salts thereof for the production of an agent for inhibiting acetyl-CoA carboxylase 2 and pyruvate dehydrogenase kinase 4.
20) A method for promoting energy consumption containing administering or ingesting chlorogenic acids or salts thereof.
21) A method for promoting fat burning containing administering or ingesting chlorogenic acids or salts thereof.
22) A method for promoting carbohydrate burning containing administering or ingesting chlorogenic acids or salts thereof.
23) A method for promoting fat burning and carbohydrate burning containing administering or ingesting chlorogenic acids or salts thereof.
24) A method for improving an effect of exercise containing administering or ingesting chlorogenic acids or salts thereof.
25) A method for improving motor functions containing administering or ingesting chlorogenic acids or salts thereof.
26) A method for inhibiting acetyl-CoA carboxylase 2 containing administering or ingesting chlorogenic acids or salts thereof.
27) A method for inhibiting pyruvate dehydrogenase kinase 4 containing administering or ingesting chlorogenic acids or salts thereof.

### BRIEF DESCRIPTION OF DRAWINGS

FIG. 1 shows graphs illustrating an average amount of energy consumed, an average amount of fat burned, and an average amount of carbohydrate burned during continuous ingestion.

FIG. 2 shows graphs illustrating an average amount of energy consumed and an average amount of fat burned by single ingestion.

FIG. 3 shows graphs illustrating the amount of energy consumed and the amount of fat burned during exercise.

FIG. 4 shows a graph illustrating an inhibitory action of chlorogenic acids on the expression of ACC2 mRNA and PDK4 mRNA.

FIG. 5 shows graphs illustrating inhibitory actions of chlorogenic acids and nine kinds of quinic acid derivatives on the expression of ACC2 mRNA and PDK4 mRNA. In the figure, CQA, FQA, and diCQA represent caffeoylquinic acid, feruloylquinic acid, and dicaffeoylquinic acid, respectively.

FIG. 6 shows a graph illustrating an effect of a packaged beverage containing chlorogenic acids on the promotion of energy consumption.

FIG. 7 shows a graph illustrating the influence of a packaged beverage containing chlorogenic acids on the anaerobic metabolism threshold.

### EMBODIMENTS FOR CARRYING OUT THE INVENTION

The present invention relates to provision of a pharmaceutical product, a quasi-drug, a food, a beverage, a feed such as a pet food, and others which are highly safe, have an excellent action of promoting energy consumption, promoting fat burning, promoting carbohydrate burning, improving the effect of exercise, or improving motor functions, and are effective for the reduction of the risk of development, the prevention or amelioration of obesity or metabolic syndrome, the improvement of motor functions, and the like.

The present inventors conducted a study on energy consumption, fat burning, carbohydrate burning, and the effect of exercise. As a result, they have found that while chlorogenic acids significantly promote energy consumption, fat burning, and carbohydrate burning, they have inhibitory actions on acetyl-CoA carboxylase 2 (ACC2) and pyruvate dehydrogenase kinase 4 (PDK4), and significantly improve the effect of exercise and motor functions.

The agent for promoting energy consumption, the agent for promoting fat burning, the agent for promoting carbohydrate burning, the agent for improving the effect of exercise, and the agent for improving motor functions of the present invention have an excellent action of promoting energy consumption, promoting fat burning, promoting carbohydrate burning, improving the effect of exercise, and improving motor functions. Thus, according to the present invention, a pharmaceutical product, a quasi-drug, a food, a beverage, a feed such as a pet food, and others for the reduction of the risk of development, the prevention or amelioration of obesity or metabolic syndrome, the improvement of the effect of exercise, and the improvement of motor functions can be provided.

In the present invention, examples of the chlorogenic acids include a compound in which one to two hydroxyl groups selected from the positions 3, 4, and 5 of quinic acid are ester-linked to coffeic acid and/or ferulic acid. Specific examples thereof include 3-caffeoylquinic acid, 4-caffeoylquinic acid, 5-caffeoylquinic acid (chlorogenic acid), 3,4-dicaffeoylquinic acid, 3,5-dicaffeoylquinic acid, and 4,5-dicaffeoylquinic acid, and a mixture of two or more thereof may also be used. Further, feruloylquinic acids such as 3-feruloylquinic acid, 4-feruloylquinic acid, and 5-feruloylquinic acid may be included.

Examples of a salt of chlorogenic acids include a salt with an alkali metal such as sodium and potassium, a salt with an alkaline earth metal such as magnesium and calcium, a salt with an organic amine such as monoethanolamine, diethanolamine, and triethanolamine, and a salt with a basic amino acid such as arginine, lysine, histidine, and ornithine. Also, the chlorogenic acids of the present invention include one containing a free carboxyl group.

The chlorogenic acids or a salt thereof may be used in form of one produced by general chemical synthesis, an extract isolated or purified from nature, or a natural product containing them directly. The extraction may be performed by an ordinary method, and examples of the extract include one derived from coffee beans, apples, grapes, onions, sweet potatoes, and the like. A coffee bean-derived extract is preferable.

Examples of a commercially available preparation containing a large amount of chlorogenic acids or a salt thereof include FLAVOR HOLDER RC-30R (T. Hasegawa. Co., Ltd.), raw coffee bean extract P (Oryza Oil & Fat Chemical Co., Ltd.), and OXCH100 (Toyo Hakko Co., Ltd.). These commercial products can be further purified to increase the purity of chlorogenic acids or a salt thereof. As the production method of chlorogenic acids or a salt thereof, for example, the methods disclosed in JP-A-2006-241006, JP-A-2006-306799, JP-A-2008-94758, JP-A-2008-94759, and the like may be employed.

In general, in vivo energy metabolism is strictly regulated and homeostasis of the amount of energy produced/balance is maintained. Thus, changes in the expression level or the activity of specific molecules are rarely reflected directly in the energy metabolism from an individual-level perspective. Therefore, a macroscale energy state at an individual level cannot be accurately understood only from the results of the expression levels of a specific gene and a specific enzyme activity. The present invention is based on a finding that, as a result of a search for a truly effective ingredient by measuring the amount of energy consumed at an individual level, chlorogenic acids are effective as an agent for promoting energy consumption, an agent for promoting fat burning, an agent for promoting carbohydrate burning, an agent for improving the effect of exercise, and an agent for improving motor functions.

In the present invention, energy consumption means that nutrients (energy sources) are metabolized in each biological tissue and converted into chemical energy or heat energy. The amount of energy consumed is calculated from the amount of oxygen consumed during that process, and it refers to the amount of physicochemical energy produced on the macroscale at an individual level. Therefore, an action of promoting energy consumption refers to an action of increasing the amount of energy consumed as defined above. Fat burning means that a fatty acid is metabolized in each biological tissue and converted into chemical energy or heat energy.

The amount of fat burned is calculated from the amount of oxygen consumed and the amount of carbon dioxide discharged during the oxidative metabolism process of fat by using, for example, the following equation of Peronnet, et al., and it indicates the amount of fat-derived energy produced at an individual level. Therefore, an action of promoting fat burning refers to an action of increasing the amount of fat burned as defined above.

Carbohydrate burning means that carbohydrate is metabolized in each biological tissue and converted into chemical energy or heat energy. The amount of carbohydrate burned is calculated from the amount of oxygen consumed and the amount of carbon dioxide discharged during that process by using, for example, the following equation of Peronnet, et al., and it indicates the amount of carbohydrate-derived energy produced at an individual level. Therefore, an action of promoting carbohydrate burning refers to an action of increasing the amount of carbohydrate burned as defined above.

The amount of fat burned=1.695×(1−1.701/1.695×respiratory quotient)×the amount of oxygen consumed The amount of carbohydrate burned=(4.585×respiratory quotient−3.226)×the amount of oxygen consumed (with the proviso that the respiratory quotient=the amount of carbon dioxide discharged/the amount of oxygen consumed)

In the present invention, an action of improving the effect of exercise refers to an effect of enhancing favorable effects inherently obtained by exercise (such as an effect of promoting energy consumption, an effect of promoting fat burning, an effect of promoting carbohydrate burning, an anti-obesity effect, and an anti-metabolic syndrome effect) to a greater extent compared to when only exercise is performed.

Also, an action of improving motor functions refers to an action of improving physical ability, for example, an aerobic metabolic ability during exercise, which is expressed as an anaerobic metabolism threshold.

In the present invention, specific examples of the action of inhibiting acetyl-CoA carboxylase 2 include inhibitory actions on the expression of ACC2 mRNA and ACC2 protein, and the ACC2 activity. ACC2 is abundantly expressed in muscle and liver, and has an action of converting acetyl CoA into malonyl CoA. From a study of an ACC2 knockout mouse, it has been revealed that inhibition of ACC2 promotes fat and carbohydrate burning to promote energy consumption (Choi C S. et al., Proc. Natl. Acad. Sci. USA, 2007; 104: 16480 to 16485). Also, combined with research findings on an agent for inhibiting ACC (Savage D B et al., J. Clin. Invest. 2006; 116: 817 to 824), etc., it can be said that an agent for inhibiting ACC2 is effective as an agent for promoting energy metabolism.

In the present invention, specific examples of the action of inhibiting pyruvate dehydrogenase kinase 4 (PDK4) include inhibitory actions on the expression of PDK4 mRNA and PDK4 protein, and the PDK4 activity. PDK4 is an enzyme responsible for switching the energy source of a living body by phosphorylation of a pyruvate complex, and inhibition of PDK4 increases the production of acetyl-CoA from glucose by enhancing the activity of a pyruvate dehydrogenase complex responsible for the decarboxylation of pyruvic acid into acetyl-CoA (Sugden, M. C., and Holness, M. J. (2003) Am. J. Physiol. Endocrinol. Metab., 284, E855 to E862). Therefore, inhibition of PDK4 is known to lead to promotion of utilization (oxidation) of carbohydrate.

These actions are fundamentally different from the previously reported fatty acid synthase-inhibiting action (an action of inhibiting fatty acid synthase, which is an enzyme responsible for fatty acid synthesis in liver and adipose tissues, in vitro), blood glucose absorption-inhibiting action (an action of inhibiting the amount of glucose absorbed when coffee containing a chlorogenic acid and sucrose are simultaneously ingested), glucose-6-phosphatase-inhibiting action (an action of inhibiting the activity of glucose-6-phosphatase, which is an enzyme responsible for glyconeogenesis, in vitro), PPAR activation action (an action of promoting PPAR-dependent gene transcription), preventive or ameliorating action on hyperinsulinemia/hyperleptinemia (an action of preventing or ameliorating hyperinsulinemia and hyperleptinemia resulting from insulin and leptin resistances), maltase-inhibiting action (an action of inhibiting maltase, which is maltose hydrolase, in vitro), and carnitine palmitoyltransferase activation action (an action of promoting the enzyme activity of liver carnitine palmitoyltransferase, which is an enzyme producing acylcarnitine from carnitine and acyl-CoA).

As will be shown in the following Examples, chlorogenic acids or salts thereof have an action of promoting energy consumption, an action of promoting fat burning, an action of promoting carbohydrate burning, an action of improving the effect of exercise, an action of improving motor functions, an ACC2-inhibiting action, and a PDK4-inhibiting action. Accordingly, the chlorogenic acids or salts thereof of the present invention can be used for a method for providing the promotion of energy consumption, the promotion of fat burning, the promotion of carbohydrate burning, the improvement of the effect of exercise, the inhibition of ACC2, the inhibition of PDK4, the prevention, amelioration, or reduction of the risk of development of obesity and metabolic syndrome, and the improvement of motor functions by injecting or administering the chlorogenic acids or salts thereof to animals including humans. Also, the chlorogenic acids or salts thereof of the present invention can be an agent for promoting energy consumption, an agent for promoting fat burning, an agent for promoting carbohydrate burning, an agent for improving the effect of exercise, an agent for improving motor functions, an agent for inhibiting ACC2, and an agent for inhibiting PDK4 (hereinbelow, referred to as “the agent for promoting energy consumption and the like”), and also, used for the production of the agent for promoting energy consumption and the like. At this point, in the agent for promoting energy consumption and the like, the chlorogenic acids or salts thereof can be used singly, or, in addition to these, substances that are acceptable to objects described below into which the chlorogenic acid or a salt thereof is to be mixed, and that include carriers appropriately selected as needed may be used. It is to be noted that this preparation can be produced by an ordinary method depending on an object into which the chlorogenic acid or a salt thereof is to be mixed. The agent for promoting energy consumption and the like can be used mixed, as an active ingredient, into a pharmaceutical product, a quasi-drug, a food, a beverage, and a feed such as a pet food, for the promotion of energy consumption, the promotion of fat burning, the promotion of carbohydrate burning, the improvement of the effect of exercise, the inhibition of ACC2, the inhibition of PDK4, the prevention, amelioration or reduction of the risk of development of obesity and metabolic syndrome, and the improvement of motor functions.

When the agent for promoting energy consumption and the like of the present invention are used as an active ingredient of a pharmaceutical product, the pharmaceutical product can be administered in any administration form. While examples of the administration form include oral administration, enteral administration, mucosal administration, and injection, oral administration is preferable. Examples of the dosage form of the preparation for oral administration include a tablet, a coated tablet, a capsule, a soft capsule, a granule, a pulvis, a powder, a sustained release preparation, a suspension, an emulsion, a troche, a liquid medicine for internal use, a sugar-coated tablet, a pill, a fine granule, a syrup, an elixir, and a milky liquid. Examples of the dosage form for parenteral administration include intravenous injection, an intramuscular injection medicine, inhalation, an infusion solution, a suppository, an inhalation medicine, a transdermal absorption medicine, an eye drop, a nasal drop, a cream, a gel, a lotion, a patch, a gel, and a paste.

Also, in the preparation, the agent for promoting energy consumption and the like of the present invention may be used singly or in combination with other pharmaceutically acceptable carriers. Examples of the carrier include an excipient, a binder, a disintegrant, a lubricant, a diluent, an osmotic pressure adjuster, a fluidity promoter, an absorption aid, a pH adjuster, an emulsifying agent, an antiseptic, a stabilizer, an antioxidant, a colorant, an ultraviolet ray absorber, a humectant, a thickener, a glazing agent, an activity enhancer, an anti-inflammatory agent, a disinfectant, a corrigent, an odor-masking agent, a filler, a surfactant, a dispersant, a buffer, a preservative, an adhesive agent, a fragrance, and a coating agent.

Specific examples of the aforementioned carrier include a solid carrier such as lactose, kaolin, sucrose, crystalline cellulose, corn starch, talc, agar, pectin, stearic acid, magnesium stearate, lecithin, and sodium chloride, and a liquid carrier such as glycerin, peanut oil, polyvinyl pyrrolidone, olive oil, ethanol, benzyl alcohol, propylene glycol, and water.

Also, an energy consumption-promoting substance such as caffeine may be further mixed into the aforementioned preparation. When caffeine is mixed in, as for a weight ratio of the content of chlorogenic acids or a salt thereof and that of caffeine, a ratio of chlorogenic acids/caffeine is preferably 20/1 to 1/1, more preferably 15/1 to 1/1, and even more preferably 10/1 to 2/1, in terms of chlorogenic acid.

Although the content of the chlorogenic acids or a salt thereof in the aforementioned preparation varies depending on the kind of the preparation, in the case of a preparation for oral administration, it is preferably 1 to 100% by weight, more preferably 5 to 95% by weight, and even more preferably 10 to 70% by weight in terms of chlorogenic acid in the total weight of the preparation.

When the agent for promoting energy consumption and the like of the present invention are used as an active ingredient of various foods and beverages, in addition to general foods and beverages, based on the concept of promotion of energy consumption, the promotion of fat burning, the promotion of carbohydrate burning, the improvement of the effect of exercise, the inhibition of ACC2, the inhibition of PDK4, the prevention, amelioration or reduction of the risk of development of obesity and metabolic syndrome, and the improvement of motor functions, they are applicable to functional foods and beverages such as cosmetic foods and beverages, foods and beverages for the sick, foods and beverages with nutrient function claims, or foods and beverages for specified health uses, displaying the aforementioned effects as needed.

The form of the food and the beverage may be solid, semi-solid, or liquid. Examples of the food and the beverage include breads, noodles, confectioneries such as cookies, snacks, jellies, milk products, frozen foods, instant foods such as powdered coffee, processed starch products, processed meat products, other processed foods, beverages such as coffee beverages, packaged beverages such as canned coffee beverages, soft drinks, and fruit juice beverages, soups, seasonings, dietary supplements, and raw materials thereof. Also, similarly to the aforementioned preparation for oral administration, the food and the beverage may be in the form of a tablet, a pill, a capsule, a solution, a syrup, a powder, a granule, and the like.

The food and the beverage in the aforementioned form can be prepared by mixing with, in addition to chlorogenic acids or a salt thereof, other food and beverage ingredients, softeners, oil, emulsifiers, antiseptics, fragrances, stabilizers, colorants, antioxidants, thickeners, adhesive agents, dispersants, humectants, and the like, and further, caffeine as described above in an appropriate combination.

Although the content of the chlorogenic acids or a salt thereof in a food and a beverage varies depending on the manner of use thereof, in the form of a beverage, it is normally 0.01 to 5% by weight, preferably 0.05 to 3% by weight, more preferably 0.1 to 2% by weight, and even more preferably 0.1 to 1% by weight in terms of chlorogenic acids. Also, in jellies, the content is normally 0.01 to 1% by weight, preferably 0.02 to 0.5% by weight, and more preferably 0.05 to 0.3% by weight. Also, in coffee beverages, the content is normally 0.01 to 1% by weight, preferably 0.05 to 1% by weight, and more preferably 0.1 to 1% by weight, and even more preferably 0.15 to 1% by weight.

Also, when the agent for promoting energy consumption and the like of the present invention are used as an active ingredient of a feed, examples thereof include a feed for livestock such as cows, pigs, chickens, sheep, and horses, a feed for small animals such as rabbits, rats, and mice, a feed for fish and shellfish such as tunas, eels, sea breams, yellowtails, and shrimps, and a pet food for dogs, cats, little birds, squirrels, and the like.

It is to be noted that when producing a feed, the agent for promoting energy consumption and the like are used singly or additionally mixed with ordinarily used feed ingredients, for example, meats such as beef, pork, and mutton, protein, grains, rice brans, sake lees, sugars, vegetables, vitamins, and minerals, and further, gelling agents, shape retaining agents, pH adjusters, seasonings, antiseptics, nutrition supplements, and the like which are ordinarily used in a feed, as needed. The above ingredients can be processed by an ordinary method to produce the feed.

No particular limitation is imposed on the dose or the intake amount of the agent for promoting energy consumption and the like of the present invention when they are used as an active ingredient of pharmaceutical products and functional foods as long as it is an effective amount. Also, although the dose or the intake amount thereof is variable depending on the condition, body weight, sex, age, or another factor of a subject, normally, the daily dose per adult for oral administration is preferably 100 to 3000 mg, more preferably 300 to 2000 mg, and even more preferably 300 to 1000 mg in terms of chlorogenic acids. Also, while the aforementioned preparation can be administered in accordance with any dosing regimen, it is preferably administered once or in several divided doses daily.

Although no particular limitation is imposed on a subject for administration or ingestion of the agent for promoting energy consumption and the like of the present invention as long as it is one in need of the agent, people suffering from obesity or metabolic syndrome or those who are likely to develop those diseases are preferable. As to the standard of obesity, in Japan, those having a BMI=25 or higher, and in Europe and America, those having a BMI=30 or higher are mainly determined to be obese. As to the diagnostic criteria for metabolic syndrome for Japanese, males having a waist circumference of 85 cm or more or females having a waist circumference of 90 cm or more who satisfy at least one item of the following three items; (1) having blood triglyceride of 150 mg/dl or more or HDL cholesterol of less than 40 mg/dl, (2) having hyperglycemia (fasting blood glucose of 110 mg/dl or more), and (3) having hypertension (130/85 mHg or more) are determined to be likely to develop metabolic syndrome, and those who satisfy two or more items are determined to have metabolic syndrome. Thus, those people are preferable as the subject of the present invention. In the U.S., those who satisfy three or more of the waist circumference (102 cm or more for males and 88 cm or more for females), high triglyceride, low HDL, hypertension, and high fasting blood glucose are determined to have metabolic syndrome; thus, those further including those likely to develop metabolic syndrome, who satisfy two or more of the above, are preferable as the subject of the present invention.

### EXAMPLES

### Production Example 1

From pulverized roasted coffee beans (2 kg), 1.45 kg of a coffee extract having a solid concentration of approximately 25% was obtained by hot water extraction. The coffee extract thus obtained was concentrated under reduced pressure to a solid concentration of approximately 35%, followed by spray drying at 195° C. to give 300 g of a powdered coffee extract. 270 g of the powdered coffee extract thus obtained was dissolved in 9 L of purified water. 9 L of the coffee solution thus obtained was allowed to flow through a 3 L column filled with a synthetic adsorbent (SEPABEADS SP70, the product of Mitsubishi Chemical Corporation). After washing the column by flowing 3 L of purified water, chlorogenic acids were eluted with 33 L of a 0.1% aqueous solution of sodium hydroxide, and then immediately neutralized to weakly acidic using ion-exchange resin (Amberlite 200 CT: Organo Corporation) and collected. The preparation liquid containing chlorogenic acids thus collected was concentrated under reduced pressure to a solid concentration of approximately 10%, followed by spray drying to give a preparation of high-purity chlorogenic acids (40 g).

The content of chlorogenic acids in the preparation of chlorogenic acids thus produced (sum of the content of each of the below-described molecular species) was 77.1%, and the composition was as follows.

3-caffeoylquinic acid (7.7%), 4-caffeoylquinic acid (15.5%), 5-caffeoylquinic acid (31.9%), 3,4-dicaffeoylquinic acid (9.0%), 3,5-dicaffeoylquinic acid (8.9%), 4,5-dicaffeoylquinic acid (5.3%), 3-feruloylquinic acid (7.6%), 4-feruloylquinic acid (5.6%), and 5-feruloylquinic acid (8.5%). It is to be noted that the preparation of chlorogenic acids thus produced did not contain caffeine.

### Production Example 2

In 1 L of hot water of 98° C., 100 g of raw coffee beans were stirred and extracted for four hours. After cooling, solid-liquid separation was performed, and the extraction liquid thus obtained was concentrated under reduced pressure to a solid concentration of 20% (w/w) at 40° C. Subsequently, the resulting product was spray dried to give a preparation of chlorogenic acids. The amount of chlorogenic acids in the coffee bean extract thus obtained was found to contain 26.3% by weight of caffeoylquinic acid (CQA), 5.1% by weight of feruloylquinic acid (FQA), and 6.65% by weight of dicaffeoylquinic acid (di-CQA).

### Production Example 3

The preparation of chlorogenic acids obtained as above was dissolved in ion-exchanged water so that the chlorogenic acids accounted for 1% by weight. After that, 2N hydrochloric acid was added to 165 g of this solution to adjust pH to 1.3, followed by centrifugation for solid-liquid separation to obtain 164 g of an upper layer part. Subsequently, 54 g of the upper layer liquid thus collected was allowed to flow through a column filled with 7.7 ml of a synthetic adsorbent (trade name SEPABEADS SP 207: the product of Mitsubishi Chemical Corporation). Subsequently, 116 g of a 0.1% sodium hydroxide solution was allowed to flow through the column, and the eluate was neutralized to weakly acidic using ion-exchange resin (Amberlite 200 CT: Organo Corporation) and collected. The preparation liquid containing chlorogenic acids thus collected was concentrated under reduced pressure to give a preparation of high-purity chlorogenic acids. The chlorogenic acids-composition thus obtained had a purity of 80% and contained 70% of monocaffeoylquinic acids, 14% of feruloylquinic acids, and 16% of dicaffeoylquinic acids.

### Production Example 4

### Preparation of Nine Kinds of Chlorogenic Acids

From the preparation of chlorogenic acids obtained in Production Example 1, nine kinds of chlorogenic acids were prepared by the medium-pressure column chromatography system (Yamazen: Ultra Pack ODS-A-40D column, UV detector PREP-UV-10V, fraction collector FR 50N, gradient mixer GR200, degassing unit, pump PUMP-600A). The preparation of chlorogenic acids (2 g) was dissolved in 20 ml of a solution A (acetic acid-methanol-water=1:20:80), applied subsequently to the column, and eluted at a flow rate of 10 ml/min while applying a gradient (0→100%) with the solution A from 0 to 100 minutes, and after that with a solution B (methanol) from 100 to 600 minutes. By this operation, 3-caffeoylquinic acid, 3,4-dicaffeoylquinic acid, 3,5-dicaffeoylquinic acid, and 4,5-dicaffeoylquinic acid were obtained as a single compound. A mixture of 4-caffeoylquinic acid, 5-caffeoylquinic acid, 3-feruloylquinic acid, 4-feruloylquinic acid, and 5-feruloylquinic acid was further purified by the preparative HPLC system (LC-908: Japan Analytical Industry Co., Ltd.) using an Inertsil ODS-3 column (GL Science Inc.). As an eluate, trifluoroacetic acid-methanol-water (1:300:700) was used at a flow rate of 9 ml/min. Elution of chlorogenic acids was monitored by measuring the absorbance at 325 nm. The structure of each compound was confirmed by ¹H-NMR (JEOL α500 NMR spectrometer: JEOL).

### Test Example 1

### Evaluation of Action of Promoting Energy Consumption, Action of Promoting Fat Burning, and Action of Promoting Carbohydrate Burning During Continuous Ingestion

C57BL/6J mice (7-week-old, male: Charles River Laboratories Japan, Inc.) were reared by using standard solid feed CE-2 (Oriental Yeast Co., Ltd.) for one week (room temperature: 23±2° C., humidity: 55±10%, a light period of 7 am to 7 pm, ad libitum water intake). The mice were then divided into two groups so that the body weight was equalized between the groups. Subsequently, the mice were administered with either a control diet (25% corn oil, 5% lard, 13% sucrose, 20% casein, 28.5% potato starch, 4% cellulose, 3.5% vitamin (trade name: AIN-76 Vitamin Mixture, Oriental Bio Service, Inc.), 1% mineral (trade name: AIN-76 Mineral Mixture, Oriental Bio Service, Inc.)) or a chlorogenic acids-containing test diet (25% vegetable oil, 5% lard, 13% sucrose, 20% casein, 27.5% potato starch, 4% cellulose, 3.5% vitamin, 1% mineral, 1% preparation of chlorogenic acids (described in Production Example 1)) for eight weeks, and then subjected to expiratory gas analysis in the ninth week.

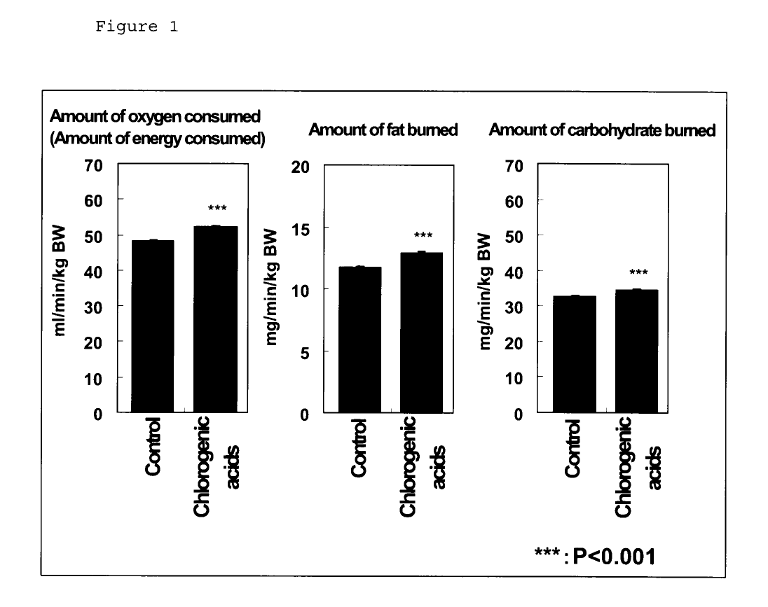

The mice were transferred to an expiratory gas analysis chamber and the expired gas was analyzed for 24 hours with Arco-2000 system (Arcosystem Inc.). In the expiratory gas analysis, the concentrations of oxygen and carbon dioxide in the chamber were measured to calculate the amount of oxygen consumed (the amount of energy consumed) by each mouse as well as the amount of fat burned and the amount of carbohydrate burned by using equation of Peronnet (Peronnet F, and Massicotte D, (1991), Can. J. Sport. Sci. 16: 23 to 29). The results of an average amount of energy consumed, an average amount of fat burned, and an average amount of carbohydrate burned are shown in FIG. 1.

In the group of mice that ingested the test diet containing the preparation of chlorogenic acids, the amount of energy consumed, the amount of fat burned, and the amount of carbohydrate burned were found to be significantly increased compared to the control group. Accordingly, chlorogenic acids are effective as an agent for promoting energy consumption, an agent for promoting fat burning, and an agent for promoting carbohydrate burning.

### Test Example 2

### Action of Promoting Energy Consumption and Action of Promoting Fat Burning by Single Ingestion

C57BL/6J mice (7-week-old, male: Charles River Laboratories Japan, Inc.) were reared by using standard solid feed CE-2 (Oriental Yeast Co., Ltd.) for one week (room temperature: 23±2° C., humidity: 55±10%, a light period of 7 am to 7 pm, ad libitum water intake). The mice were then divided into two groups so that the body weight was equalized between the groups. After eight hours of fasting, under ether anesthesia, the mice were orally administered with either a test aqueous solution containing the preparation of chlorogenic acids produced in Production Example 1 (2% (w/v) total chlorogenic acids) or a control water without chlorogenic acids at 10 ml/kg body weight, followed by oral administration of a sugar/fat mixed emulsion prepared by the following method at 20 ml/kg body weight. The mice were then immediately transferred to an expiratory gas analysis chamber and the expired gas was analyzed for one hour with Arco-2000 system (Arcosystem Inc.). In the expiratory gas analysis, the concentrations of oxygen and carbon dioxide in the chamber were measured to calculate the amount of oxygen consumed (the amount of energy consumed) by each mouse as well as the amount of fat burned by using equation of Peronnet (Peronnet F, and Massicotte D, (1991), Can. J. Sport. Sci. 16: 23 to 29).

### <Preparation of Sugar/Fat Mixed Emulsion>

Sucrose was dissolved in water to prepare a sucrose solution in advance, to which lecithin (trade name: lecithin, made from eggs, Wako Pure Chemical Industries, Ltd.) and corn oil were added according to the composition shown below. The total volume was brought to 10 ml, and the mixture was emulsified by sonication to give an emulsion.

TABLE 1

| | | |
|---|---|---|
| Sucrose | 2 | g |
| Corn oil | 2 | g |
| Lecithin | 0.08 | g |
| DW | an amount to bring the total volume to 10 | ml |

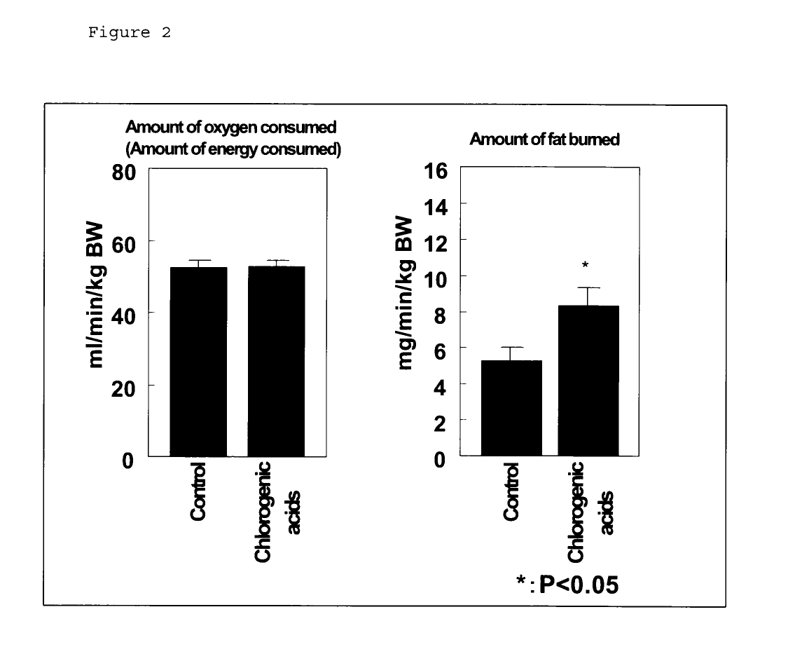

The results of an average amount of energy consumed (amount of oxygen consumed) and an average amount of fat burned are shown in FIG. 2.

The amount of fat burned was found to be significantly higher in the chlorogenic acids-administered group. Accordingly, the chlorogenic acids of the present invention are effective as an agent for promoting fat burning. Also, from this experiment, it is understood that chlorogenic acids are effective for promoting dietary fat burning.

### Test Example 3

### Action of Promoting Energy Consumption, Action of Promoting Fat Burning, and Action of Improving the Effect of Exercise During Exercise

C57BL/6J mice (6-week-old, male: Charles River Laboratories Japan, Inc.) were reared by using standard solid feed CE-2 (Oriental Yeast Co., Ltd.) for one week (room temperature: 23±2° C., humidity: 55±10%, a light period of 7 am to 7 pm, ad libitum water intake) to acclimatize them to the environment. At seven weeks old, the mice underwent 5-day training as follows to get familiarized with treadmill running exercise.
Day 1: 5 m/min (10 min)→10 m/min (10 min)→15 m/min (10 min)
Day 2: 10 m/min (10 min)→15 m/min (10 min)→20 m/min (10 min)
Day 3: 15 m/min (10 min)→20 m/min (10 min)→25 m/min (10 min)
Day 4: 15 m/min (10 min)→20 m/min (10 min)→25 m/min (10 min)
Day 5: 15 m/min (5 min)→20 m/min (5 min)→25 m/min (20 min)

At eight weeks old, the mice were divided into a control diet group (Cont) and a chlorogenic acid group so that the body weight was equalized between the groups. Subsequently, the mice were given either a control diet (5% corn oil, 20% casein, 66.5% potato starch, 4% cellulose, 3.5% vitamin (trade name: AIN-76 Vitamin Mixture, Oriental Bio Service, Inc.), 1% mineral (trade name: AIN-76 Mineral Mixture, Oriental Bio Service, Inc.)) or a test diet containing chlorogenic acids (5% corn oil, 20% casein, 65.5% potato starch, 4% cellulose, 3.5% vitamin, 1% mineral, 1% preparation of chlorogenic acids (described in Production Example 1)) for eight weeks. Also, during this rearing period, the mice underwent running training at 18 m/min (30 min) three times a week.

In the eighth week after initiation of ingestion of the test diet, gas expired during exercise was measured. A measurement test for gas expired during exercise was performed with Arco2000-system (Arcosystem Inc.). The mice were put into treadmill-type chambers for measurement of gas expired during exercise, one mouse per chamber, and kept at rest for two hours to get familiarized with the environment. Subsequently, treadmill was started, and gas expired during exercise was measured for 30 minutes. The treadmill program included 10 m/min (5 min), followed by 15 m/min (5 min), and finally 18 m/min (20 min). A respiratory exchange ratio (RER) was obtained from the amount of oxygen consumed and the amount of carbon dioxide discharged thus measured, and the amount of fat burned was calculated by using equation of Peronnet et al (Peronnet F, and Massicotte D, (1991), Can. J. Sport. Sci. 16: 23 to 29). It is to be noted that when mice refused to run or became incapable of running during the measurement, the measurement was discontinued and the mice were excluded.

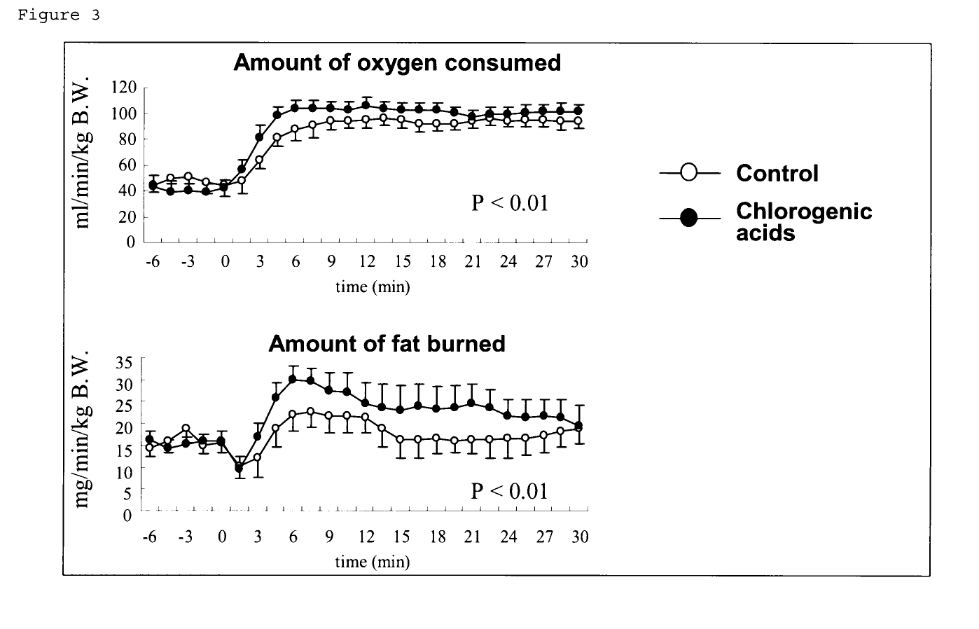

The results of the amount of energy consumed (the amount of oxygen consumed) and the amount of fat burned during exercise are shown in FIG. 3. The amount of energy consumed and the amount of fat burned are found to be significantly higher in the group of mice that ingested the test diet containing the chlorogenic acids. Accordingly, the chlorogenic acids of the present invention are effective as an agent for promoting energy metabolism and an agent for promoting fat burning. Also, from this experiment, it is understood that chlorogenic acids are effective for promoting accumulated body fat burning.

Further, while exercise increases energy consumption in general, as understood from the above results, chlorogenic acids further increases the amount of fat burned and the amount of energy consumed when used in combination with exercise. Thus, chlorogenic acids have an action of further enhancing the effect of exercise, and are considered to be effective also as an agent for enhancing the effect of exercise. This further indicates that, when exercise is performed for the prevention or amelioration of obesity and metabolic syndrome, one can achieve the goal more effectively by using the chlorogenic acids of the present invention in combination.

### Test Example 4

### Regulatory Action of Chlorogenic Acids on the Expression of Energy Metabolism-Related Molecule in Mouse

C57BL/6J mice (7-week-old, male: Charles River Laboratories Japan, Inc.) were reared by using standard solid feed CE-2 (Oriental Yeast Co., Ltd.) for one week (room temperature: 23±2° C., humidity: 55±10%, a light period of 7 am to 7 pm, ad libitum water intake). The mice were then divided into two groups so that the body weight was equalized between the groups. Subsequently, the mice were given either a control diet (25% corn oil, 5% lard, 13% sucrose, 20% casein, 28.5% potato starch, 4% cellulose, 3.5% vitamin (trade name: AIN-76 Vitamin Mixture, Oriental Bio Service, Inc.), 1% mineral (trade name: AIN-76 Mineral Mixture, Oriental Bio Service, Inc.)) or a test diet containing chlorogenic acids (25% vegetable oil, 5% lard, 13% sucrose, 20% casein, 27.5% potato starch, 4% cellulose, 3.5% vitamin, 1% mineral, and 1% preparation of chlorogenic acids (described in Production Example 1)) for two weeks. The mice were then dissected and the liver was taken. After preparing total RNA according to a standard method, cDNA was prepared using SuperScript first-strand synthesis system (Invitrogen). Quantitative PCR was performed on the cDNA thus obtained by ABI PRISM 7500 Sequence Detection System (Applied Biosystems) using Power SYBR Green Master Mix (Applied Biosystems). Also, the expression level of each gene was corrected using the expression level of 36B4 as an internal standard, and represented as a relative expression level by setting the expression level of control at 100. The primer sequences used for PCR are as shown below.

| | |
|---|---|
| ACC2-F: ACGAGCACACACAGTCCATG | (SEQ ID NO: 1) |
| ACC2-R: GATGACCTCTGGATGTTCTTG | (SEQ ID NO: 2) |
| 36B4-F: CTGATCATCCAGCAGGTGTT | (SEQ ID NO: 3) |
| 36B4-R: CCAGGAAGGCCTTGACCTTT | (SEQ ID NO: 4) |
| PDK4-F: AGGGAGGTCGAGCTGTTCTC | (SEQ ID NO: 5) |
| PDK4-R: GGAGTGTTCACTAAGCGGTCA | (SEQ ID NO: 6) |

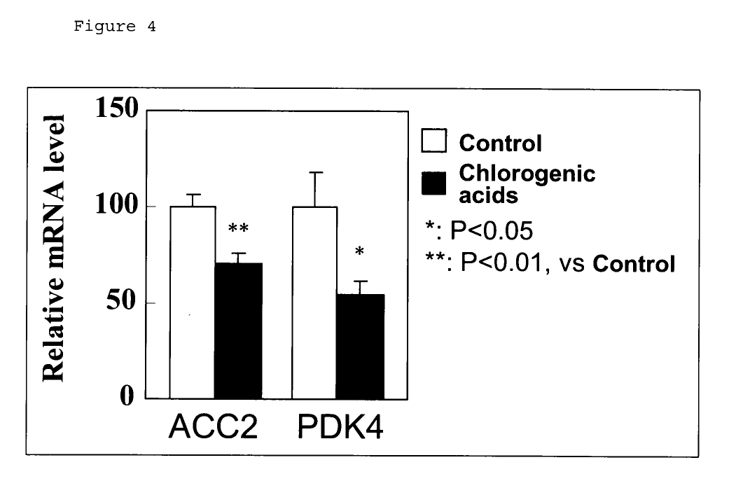

The results are shown in FIG. 4. In the liver of mice that ingested chlorogenic acids, the expression of ACC2 mRNA, which negatively regulates energy metabolism, was found to be significantly low. Because inhibition of ACC2 leads to promotion of energy consumption, fat burning, and carbohydrate burning, chlorogenic acids are considered to be effective as an agent for promoting energy metabolism. Also, in mice that ingested chlorogenic acids, the expression of PDK4 mRNA, which negatively regulates carbohydrate metabolism, was found to be significantly low. Thus, it is understood that chlorogenic acids have an action of promoting carbohydrate burning and energy metabolism.

### Test Example 5

### Regulatory Action of Chlorogenic Acids on the Expression of Energy Metabolism-Related Molecule in Liver Cell

A cultured mouse liver cell line (Hepa1-6 cell) was cultured in 10% bovine serum-containing Dulbeco's Modified Eagle's Medium (DMEM) at 37° C., 5% CO₂. The cells were seeded in a 6-well plate, and when they reached subconfluency, the medium was replaced by a serum free medium (DMEM, -FBS), followed by culturing for further 12 hours. Subsequently, various samples were added (final concentration: chlorogenic acids (Production Example 1) 2.5×10⁻⁴% (w/v) and each of the chlorogenic acids (Production Example 4) 5 mM), and after 24 hours of culturing, total RNA was prepared according to a standard method. After preparing cDNA by reverse transcription reaction, quantitative PCR was performed by ABI PRISM 7000 Sequence Detection System (Applied Biosystems) using Power SYBR Green Master Mix (Applied Biosystems). Also, the expression level of each gene was corrected using the expression level of 36B4 as an internal standard, and represented as a relative expression level by setting the expression level of control at 100.

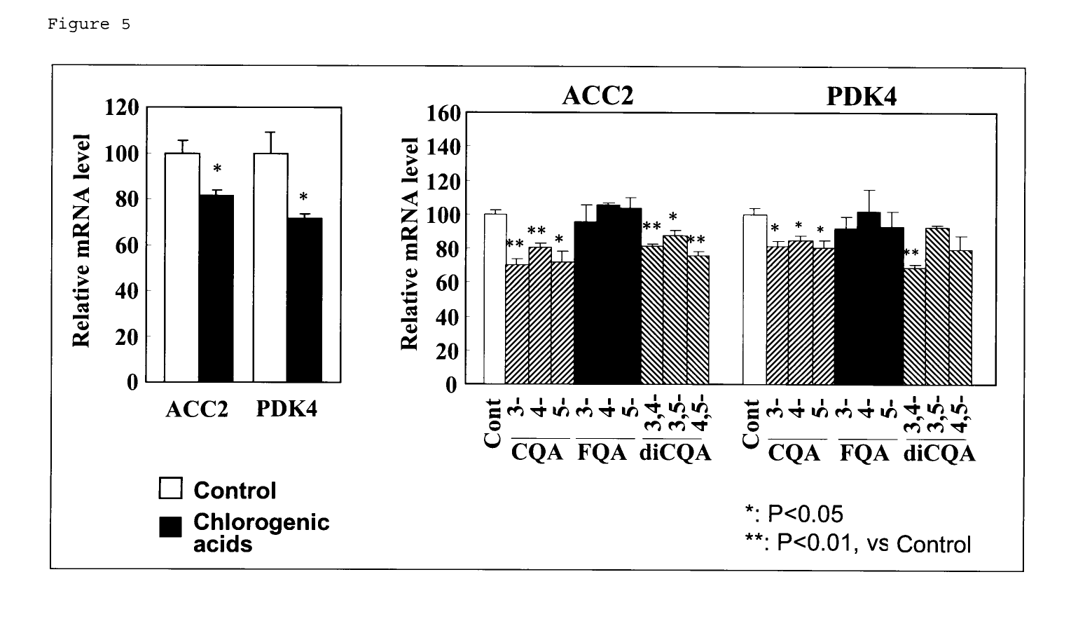

The results are shown in FIG. 5. Chlorogenic acids significantly inhibited the expression of ACC2 mRNA, which negatively regulates energy metabolism, and the expression of PDK4 mRNA, which negatively regulates carbohydrate metabolism. Also, as a result of a similar evaluation of nine kinds of chlorogenic acids contained in the preparation of chlorogenic acids (3-caffeoylquinic acid (3-CQA), 4-caffeoylquinic acid (4-CQA), 5-caffeoylquinic acid (5-CQA), 3,4-dicaffeoylquinic acid (3,4-diCQA), 3,5-dicaffeoylquinic acid (3,5-diCQA), 4,5-dicaffeoylquinic acid (4,5-diCQA), 3-feruloylquinic acid (3-FQA), 4-feruloylquinic acid (4-FQA), and 5-feruloylquinic acid (5-FQA)), six kinds of chlorogenic acids of 3-caffeoylquinic acid, 4-caffeoylquinic acid, 5-caffeoylquinic acid, 3,4-dicaffeoylquinic acid, 3,5-dicaffeoylquinic acid, and 4,5-dicaffeoylquinic acid significantly reduced the expression of ACC2 mRNA. Because inhibition of ACC2 leads to promotion of energy consumption, fat burning, and carbohydrate burning, chlorogenic acids, particularly the aforementioned six compounds are considered to be effective as an agent for promoting energy metabolism, an agent for promoting fat burning, and an agent for promoting carbohydrate burning. Also, the expression of PDK4 mRNA was significantly inhibited by 3-caffeoylquinic acid, 4-caffeoylquinic acid, 5-caffeoylquinic acid, and 3,4-dicaffeoylquinic acid. Because inhibition of PDK4 leads to promotion of utilization of carbohydrate, chlorogenic acids, particularly the aforementioned four compounds are considered to be effective as an agent for promoting energy metabolism and an agent for promoting carbohydrate burning.

### Test Example 6

### Effect of Packaged Beverage Containing Chlorogenic Acids on the Promotion of Energy Consumption in Human

Using a packaged coffee beverage containing chlorogenic acids as a test beverage and a packaged coffee-like beverage without chlorogenic acids as a control beverage, the effect of chlorogenic acids on the promotion of energy metabolism in humans was verified. This study was performed as a double-blind cross-over study.

Test beverage (185 ml): chlorogenic acids (3-, 4-, 5-caffeoylquinic acid, total: 306 mg (0.165% by weight)), hydroxyhydroquinone: 0.11 mg, calories: 24 kcal

Control beverage (185 ml): chlorogenic acids (0 mg), hydroxyhydroquinone: 0.006 mg, calories: 24 kcal

Seven subjects were divided into two groups and drank either one of the above beverages for one week, one bottle daily. After a washout period of one week, each subject drank the other beverage for one week, one bottle daily. After ingesting each beverage for one week, expiratory gas analysis was performed (the amount of oxygen consumed (VO2) and the amount of carbon dioxide discharged (VCO2) were measured) using an expiration analyzer (ARCO2000: Arcosystem Inc.) to evaluate the amount of energy consumed.

The expiratory gas analysis was performed as follows:

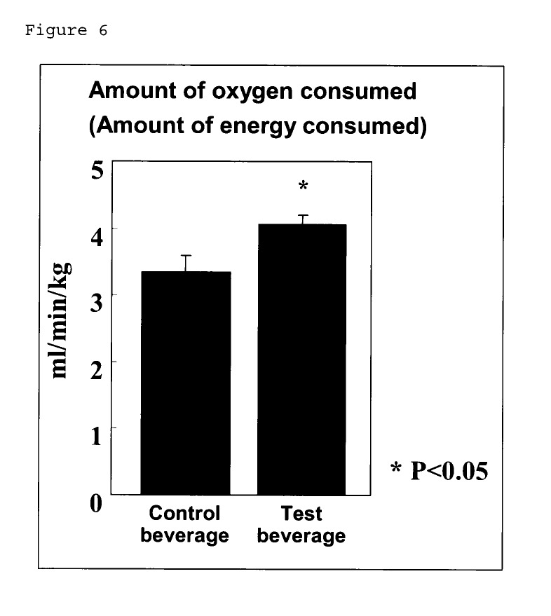

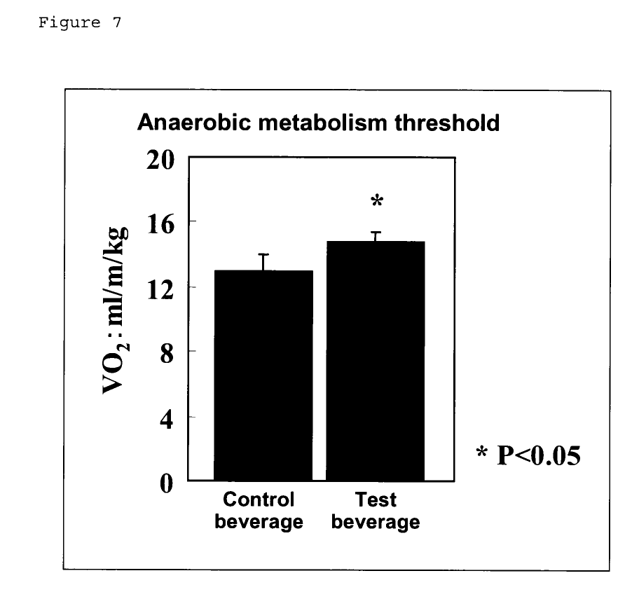

After the subjects were left at rest for 15 minutes and got familiarized with the environment, they ingested test products (either a test beverage or a control beverage) and sandwiches (540 kcal). After 30 minutes, their expiration was analyzed while at rest (for 15 minutes). Thereafter, expired gas was analyzed every 30 minutes, for 15 minutes each time (the results of measurement of an average amount of oxygen consumed (amount of energy consumed) up to two hours are shown in FIG. 6). Four hours after ingestion of the test products, the subjects warmed up for three minutes by using an ergometer (20 W, 3 minutes) for exercise, and expired gas during exercise was analyzed while gradually increasing an exercise load up to 60% of the maximal heart rate (maximal heart rate=220−age (Achtew, Metabolism, 52: 747 to 752, 2003)) at 15 W/min. The results of anaerobic metabolism threshold (anaerobic threshold: AT) calculated from the results of expiratory gas analysis are shown in FIG. 7.

As shown in FIG. 6, compared to the control beverage group, the amount of oxygen consumed (energy consumed) in the test beverage group was significantly higher, based on which the effect of the packaged beverage containing chlorogenic acids of the present invention on the promotion of energy consumption was verified.

The anaerobic metabolism threshold refers to an exercise intensity serving as a changing point at which a state in which muscle receives a sufficient oxygen supply shifts to a state in which muscle receives inadequate oxygen, when one is exercising in a way that the exercise intensity is gradually increased. Namely, the anaerobic metabolism threshold refers to the uppermost exercise intensity of aerobic exercise. As shown in FIG. 7, as the anaerobic metabolism threshold was significantly high in the test beverage group, it is understood that the aerobic metabolic ability during exercise, namely, the motor function is improved by ingestion of the packaged beverage containing chlorogenic acids of the present invention.

As shown above, the chlorogenic acids of the present invention has an action of promoting energy consumption, an action of promoting fat burning, an action of promoting carbohydrate burning, an action of improving the effect of exercise, an ACC2-inhibiting action, a PDK4-inhibiting action, and an action of improving motor functions.

### Example 1

### Capsule

The following compositions (400 mg) were encapsulated in capsules.

TABLE 2

| | |
|---|---|
| 5-Caffeoylquinic acid | 60% by weight |
| Corn starch | 20% by weight |
| Cellulose | 10% by weight |
| Lactose | 10% by weight |

### Example 2

### Soft Capsule

Soft capsule films were filled with chlorogenic acids (Production Example 1) to produce soft capsules.

TABLE 3
<Soft capsule film composition>

| | |
|---|---|
| Gelatin | 70.0% by weight |
| Glycerin | 22.9% by weight |
| Methyl parahydroxybenzoate | 0.15% by weight |
| Propyl parahydroxybenzoate | 0.51% by weight |
| Water | balance |

<Filling composition>

| | |
|---|---|
| Corn oil | 75% by weight |
| Chlorogenic acids (Production Example 1) | 25% by weight |

### Example 3

### Tablet

Each of the following components was mixed and then tableted to produce tablets.

TABLE 4

| | |
|---|---|
| Chlorogenic acids described in | 50% by weight |
| Production Example 2 | |
| Lactose | 10% by weight |
| Glucose | 2% by weight |
| Starch | 38% by weight |
| Fragrance | trace |

### Example 4

### Tablet

Each of the following components was mixed and then tableted to produce tablets.

TABLE 5

| | |
|---|---|
| Chlorogenic acids described | 50% by weight |
| in Production Example 1 | |
| Lactose | 10% by weight |
| Glucose | 2% by weight |
| Starch | 33% by weight |
| Caffeine | 5% by weight |
| Fragrance | trace |

### Example 5

### Tablet

Each of the following components was mixed and then tableted to produce tablets.

TABLE 6

| | |
|---|---|
| Raw coffee bean extract (the product of Oryza Oil & Fat | 10% by weight |
| Chemical Co., Ltd.) | |
| Chlorogenic acids described in Production Example 1 | 10% by weight |
| Lactose | 20% by weight |
| Glucose | 10% by weight |
| Starch | 50% by weight |
| Fragrance | trace |

### Example 6

### Tablet

Each of the following components was mixed and then tableted to produce tablets.

TABLE 7

| | |
|---|---|
| 5-Caffeoylquinic acid (chlorogenic acid) | 30% by weight |
| Lactose | 15% by weight |
| Glucose | 10% by weight |
| Starch | 30% by weight |
| Vitamin C | 15% by weight |
| Fragrance | trace |

### Example 7

### Tablet

Each of the following components was mixed and then tableted to produce tablets.

TABLE 8

| | |
|---|---|
| Chlorogenic acids described | 25% by weight |
| in Production Example 3 | |
| Lactose | 30% by weight |
| Glucose | 10% by weight |
| Starch | 24% by weight |
| Vitamin C | 5% by weight |
| Vitamin E | 1% by weight |
| Fructose | 5% by weight |
| Fragrance | trace |

### Example 8

### Canned Coffee Beverage

A coffee composition containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention were produced as follows.

In water, 5 g of caffeineless instant coffee (NESCAFE: the product of Nestle Japan Ltd.) was dissolved, and the resulting solution was subjected to a column filled with 1 kg of ODS fillers (octadecylsilylated silica gel) (YMC GEL ODS-A, a pore diameter of 6 nm and a particle diameter of 150 μm). After washing with 12 L of a 0.5% aqueous solution of acetic acid, chlorogenic acids were eluted with 12 L of methanol and then concentrated under reduced pressure to give 1.8 g of a preparation of chlorogenic acids. The content of chlorogenic acids in this preparation was 14.5%. Also, hydroxyhydroquinone, a kind of oxidative components, was not contained. Subsequently, the preparation of chlorogenic acids thus obtained was dissolved in 190 mL of water and packed in cans, and subjected to sterilization at 121° C. for 10 minutes to produce canned coffee beverages.

### Example 9

### Canned Coffee Beverage

A canned coffee beverage containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention were produced as follows.

Ten grams of the preparation of chlorogenic acids produced according to Production Example 1 was dissolved in 3 L of water and then packed in 190 mL steel cans. Using a retort sterilizer, the resulting products were subjected to heat treatment at 121.5° C. for 10 minutes under such a condition that the F value (sterilization index) was 10 to produce canned coffee beverages.

### Example 10

### Canned Coffee Beverage

To pulverized roasted coffee beans, eight parts hot water was added to obtain a coffee extract. This coffee extract was allowed to flow through a column filled with an activated carbon in an amount of 50% by weight with respect to the Brix of the coffee extract thus obtained (SHIRASAGI: Japan EnviroChemicals, Ltd.) at 25° C. for activated carbon treatment to give a coffee composition from which an oxidative component (hydroxyhydroquinone) was removed. The content of chlorogenic acids in the coffee composition thus obtained was measured, and after diluting with ion-exchanged water, pH of the resulting solution was adjusted with sodium bicarbonate. Then, 190 g of the coffee composition thus obtained was packed, subsequently sealed in cans and subjected to retort sterilization to give canned coffee beverages containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention. The content of chlorogenic acids was 0.176%.

### Example 11

### Canned Coffee Beverage

To pulverized roasted coffee beans, eight parts hot water was added to obtain a coffee extract. To this coffee extract (10 L), FLAVOR HOLDER (FH-1242, T. Hasegawa. Co., Ltd.) (100 mL) and water (80 L) were added, and pH was adjusted with sodium bicarbonate. The resulting solution was packed in 190 mL steel cans. After packing into cans, retort sterilization (124° C., 20 minutes) was performed. The content of chlorogenic acids was 0.13%.

### Example 12

### Powdered Coffee

Powdered coffee containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention was produced as follows. To pulverized roasted coffee beans, eight parts hot water was added to obtain a coffee extract. Then, 1 g of the preparation of chlorogenic acids of Production Example 1 was added per L of the coffee extract, and the resulting solution was then freeze-dried to give powdered coffee.

### Example 13

One gram of the preparation of chlorogenic acids produced according to Production Example 1 was dissolved per L of water. The resulting solution was sterilized by filtration and then packed in 500 mL PET bottles to produce soft drinks containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4. The products thus obtained are palatable enough for continuous consumption.

### Example 14

### Canned Coffee Beverage

A coffee composition containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention were produced as follows.

Ten grams of the preparation of chlorogenic acids produced according to Production Example 1 was dissolved in 3 L of a roasted coffee extract and then packed in 190 mL steel cans. Using a retort sterilizer, the resulting products were subjected to heat treatment at 121.5° C. for 10 minutes to produce canned coffee beverages.

### Example 15

The preparation of chlorogenic acids produced according to Production Example 1 was dissolved in water in accordance with the compositions shown below. After sterilization by filtration, the resulting solution was packed in 100 mL brown bottles to produce a soft drink composition containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention.

TABLE 9

| | |
|---|---|
| Chlorogenic acids described | 0.75% by weight |
| in Production Example 1 | |
| Vitamin B1 | 0.005% by weight |
| Vitamin B2 | 0.005% by weight |
| Vitamin B6 | 0.005% by weight |
| Taurine | 1% by weight |
| Citric acid | 0.05% by weight |
| Caffeine anhydrous | 0.05% by weight |
| Inositol | 0.05% by weight |
| Fragrance | trace |

### Example 16

Each of the following components was dissolved in water. After sterilization by filtration, the resulting solution was packed in 100 mL brown bottles to produce soft drink containing the agent for promoting energy consumption, the agent for promoting fat burning, the agent for promoting carbohydrate burning, the agent for improving the effect of exercise, the agent for improving motor functions, the agent for inhibiting ACC2, and the agent for inhibiting PDK4 containing the chlorogenic acids of the present invention.

TABLE 10

| | |
|---|---|
| 3-Caffeoylquinic acid | 0.01% by weight |
| 4-Caffeoylquinic acid | 0.01% by weight |
| 5-Caffeoylquinic acid | 0.2% by weight |
| 3,4-Caffeoylquinic acid | 0.01% by weight |
| 3,5-Caffeoylquinic acid | 0.01% by weight |
| 4,5-Caffeoylquinic acid | 0.01% by weight |
| Vitamin B1 | 0.005% by weight |
| Vitamin B2 | 0.005% by weight |
| Vitamin B6 | 0.005% by weight |
| Taurine | 1% by weight |
| Citric acid | 0.05% by weight |
| Caffeine anhydrous | 0.05% by weight |
| Inositol | 0.05% by weight |
| Fragrance | trace |

### Example 17

### Tablet

Each of the following components was mixed and then tableted to produce tablets.

TABLE 11

| | |
|---|---|
| 5-Caffeoylquinic acid | 25% by weight |
| 3,4-Caffeoylquinic acid | 2% by weight |
| 3,5-Caffeoylquinic acid | 2% by weight |
| 4,5-Caffeoylquinic acid | 2% by weight |
| Lactose | 15% by weight |
| Glucose | 10% by weight |
| Starch | 34% by weight |
| Vitamin C | 10% by weight |
| Fragrance | trace |

1-21. (canceled)

22. A method, comprising administering, to a subject in need thereof, or ingesting a chlorogenic acid or a salt thereof, thereby
promoting energy consumption in said subject;
promoting fat burning in said subject;
promoting carbohydrate burning in said subject;
promoting fat burning and carbohydrate burning in said subject;
improving an effect of exercise in said subject;
improving motor functions in said subject;
inhibiting acetyl-CoA carboxylase 2 in said subject;
inhibiting pyruvate dehydrogenase kinase 4 in said subject; or
inhibiting acetyl-CoA carboxylase 2 and pyruvate dehydrogenase kinase 4 in said subject.

23-29. (canceled)

30. The method according to claim 22, wherein the chlorogenic acid comprises at least one member selected from the group consisting of 3-caffeoylquinic acid; 4-caffeoylquinic acid; 5-caffeoylquinic acid; 3,4-dicaffeoylquinic acid; 3,5-dicaffeoylquinic acid; and 4,5-dicaffeoylquinic acid.

31. The method according to claim 22, wherein the chlorogenic acid comprises at least one member selected from the group consisting of 3-caffeoylquinic acid; 4-caffeoylquinic acid; 5-caffeoylquinic acid; and 3,4-dicaffeoylquinic acid.
